SEQUENCE LISTING (2 sequences)

SEQ ID NO: 1 - protein, 681 aa, Homo sapiens
  GFAT-alpha; GenBank accession No. M90516
MCGIFAYLNYHVPRTRREILETLIKGLQRLEYRGYDSAGVGFDGGNDKDWEANACKTQLIKKKGKVKALDEEVHKQQDMDLDIEFDVHLGIAHTRWATHGEPSPVNSHPQRSDKNNEFIVIHNGIITNYKDLKKFLESKGYDFESETDTETIAKLVKYMYDNRESQDTSFTTLVERVIQQLEGAFALVFKSVHFPGQAVGTRRGSPLLIGVRSEHKLSTDHIPILYRTGKDKKGSCNLSRVDSTTCLFPVEEKAVEYYFASDASAVIEHTNRVIFLEDDDVAAVVDGRLSIHRIKRTAGDHPGRAVQTLQMELQQIMKGNFSSFMQKEIFEQPESVVNTMRGRVNFDDYTVNLGGLKDHIKEIQRCRRLILIACGTSYHAGVATRQVLEELTELPVMVELASDFLDRNTPVFRDDVCFFLSQSGETADTLMGLRYCKERGALTVGITNTVGSSISRETDCGVHINAGPEIGVASTKAYTSQFVSLVMFALMMCDDRISMQERRKEIMLGLKRLPDLIKEVLSMDDEIQKLATELYHQKSVLIMGRGYHYATCLEGALKIKEITYMHSEGILAGELKHGPLALVDKLMPVIMIIMRDHTYAKCQNALQQVVARQGRPVVICDKEDTETIKNTKRTIKVPHSVDCLQGILSVIPLQLLAFHLAVLRGYDVDFPRNLAKSVTVE

SEQ ID NO: 2 - protein, 682 aa, Homo sapiens
  GFAT-beta; GenBank accession No. AB016789
MCGIFAYMNYRVPRTRKEIFETLIKGLQRLEYRGYDSAGVAIDGNNHEVKERHIQLVKKRGKVKALDEELYKQDSMDLKVEFETHFGIAHTRWATHGVPSAVNSHPQRSDKGNEFVVIHNGIITNYKDLRKFLESKGYEFESETDTETIAKLIKYVFDNRETEDITFSTLVERVIQQLEGAFALVFKSVHYPGEAVATRRGSPLLIGVRSKYKLSTEQIPILYRTCTLENVKNICKTRMKRLDSSACLHAVGDKAVEFFFASDASAIIEHTNRVIFLEDDDIAAVADGKLSIHRVKRSASDDPSRAIQTLQMELQQIMKGNFSAFMQKEIFEQPESVFNTMRGRVNFETNTVLLGGLKDHLKEIRRCRRLIVIGCGTSYHAAVATRQVLEELTELPVMVELASDFLDRNTPVFRDDVCFFISQSGETADTLLALRYCKDRGALTVGVTNTVGSSISRETDCGVHINAGPEIGVASTKAYTSQFISLVMFGLMMSEDRISLQNRRQEIIRGLRSLPELIKEVLSLEEKIHDLALELYTQRSLLVMGRGYNYATCLEGALKIKEITYMHSEGILAGELKHGPLALIDKQMPVIMVIMKDPCFAKCQNALQQVTARQGRPIILCSKDDTESSKFAYKTIELPHTVDCLQGILSVIPLQLLSFHLAVLRGYDVDFPRNLAKSVTVE

(19) United States
(12) Patent Application Publication
(10) Pub. No.: US 2004/0191811 A1
(43) Pub. Date: Sep. 30, 2004

(54) Methods for measuring levels of uridine-diphosphate-N-acetylglucosamine and activity of inhibitors thereof

(76) Inventors: Charles Burghardt, Mahwah, NJ (US); Jarema Peter Kochan, Towaco, NJ (US)

Correspondence Address: HOFFMANN-LA ROCHE INC., PATENT LAW DEPARTMENT, 340 KINGSLAND STREET, NUTLEY, NJ, 07110

(21) Appl. No.: 10/741,202

(22) Filed: Dec. 19, 2003

Related U.S. Application Data
(60) Provisional application No. 60/434,534, filed on Dec. 19, 2002.

(51) Int. Cl. C12Q 1/68; G01N 33/00

(52) U.S. Cl. 435/6; 436/94

(57) ABSTRACT

The present relates to methods for measuring the formation of uridine-diphosphate-N-acetylglucosamine in extracts from human and animal tissue or cells in tissue cultures. The present invention also relates to methods for measuring the inhibitory activity of a test compound on uridine-diphosphate-N-acetylglucosamine formation in cells. The present invention further relates to methods for measuring the inhibitory activity of a test compound on uridine-diphosphate-N-acetylglucosamine formation in a human or an animal.

### PRIORITY TO RELATED APPLICATIONS

This application claims the benefit of U.S. Provisional Application Serial No. 60/434,534, filed Dec. 19, 2002.

### FIELD OF THE INVENTION

The present invention pertains to methods for measuring the activity of glutamine:fructose 6-phosphate amidotransferase and to methods for measuring the inhibitory activity of a test compound on glutamine:fructose 6-phosphate amidotransferase. The present invention also pertains to methods for measuring the formation of uridine-diphosphate-N-acetylglucosamine in extracts from human and animal tissue or cells in tissue cultures, to methods for measuring the inhibitory activity of a test compound on uridine-diphosphate-N-acetylglucosamine formation in cells, and to methods for measuring the inhibitory activity of a test compound on uridine-diphosphate-N-acetylglucosamine formation in a human or an animal.

### BACKGROUND OF THE INVENTION

Methods for analyzing glucosamine 6-phosphate and uridine-diphosphate-N-acetylglucosamine (UDP-N-acetylglucosamine, UDP-GlcNAc) are known. Glucosamine 6-phosphate is the first product of the enzyme glutamine:fructose 6-phosphate amidotransferase (GFAT), which is the rate-limiting enzyme in the hexosamine biosynthetic pathway. UDP-N-acetylglucosamine is the final product in the hexosamine biosynthetic pathway, which is then used, in the N-linked and O-linked glycosylation of proteins. In addition, it is acted upon by O-N-acetylglucosamine transferase to transfer a single N-acetylglucosamine residue onto serine or threonine amino acids. These pathways are important in the regulation of protein glycosylation and have been associated with insulin sensitivity and glucose regulation. The measurement of glucosamine 6-phosphate has been labor intensive using either of two methods for detection: 1) extraction and purification of the sample followed by HPLC/MS analysis (K. A. Robinson, M. L. Weinstein, G. E. Lindenmayer, and M. G. Buse (1995) Diabetes 44 1438-1446); or 2) a spectrophotometric method that requires either boiling the samples in water, or extracting the samples with perchloric acid followed by neutralization, and centrifugation steps. These methods are not amenable to high throughput assays.

This method of glucosamine 6-phosphate determination is used to identify GFAT inhibitors as well as their subsequent optimization. The measurement of UDP-GlcNAc is used to determine the effect of GFAT inhibitors in vivo, as well as the effects of various modulators of the enzymes in the hexosamine pathway.

The methods for analyzing glucosamine 6-phosphate and UDP-N-acetylglucosamine are based on the color reaction of N-acetylglucosamine with p-dimethylaminobenzaldehyde (Ehrlich's reagent) in dilute alkali solution (W. J. Morgan and L. A. Elson (1934) Biochem J. 28, 988-995). Modifications of this original procedure using a borate buffer for a more stable pH increased the yield by 2 fold and reduced the time of color development but also required high heat and ml quantities of reagents (J. L. Reissig, J. L. Strominger, and L. F. LeLoir (1955) JBC 217, 959-966). Acetylation of glucose 6-phosphate using a dilute acetic anhydride acetone solution followed by a borate alkali solution enabled quantitative conversion of glucosarnine into N-acetylglucosamine but also required ml volumes and heating in boiling water (G. A. Levvy and A. McAllan (1959) Biochem J 73, 127-132). This method is capable of measuring glucosamine 6-phosphate in various tissues but requires the heating step in capped tubes (T. C. Richards and O. Greengard (1973) Biochemica et Biophysica Acta 304, 842-850). Others have measured GFAT activity in smaller quantities but the method requires perchloric acid to stop the reaction, neutralization with potassium hydroxide, centrifugation, and transfer of the supernatant for analysis (G. L. McKnight, S. L. Mudri, S. L. Mathewest, R. R. Traxinger, S. Marshall, P. O. Sheppard, and P. J. O'Hara (1992), JBC 267, 25204-25212). A spectrophotometric method for quantitation of UDP-N-acetylglucosamine has not been described. The current methods require purification of the sample and analysis by HPLC/MS (K. A. Robinson, M. L. Weinstein, G. E. Lindenmayer, and M. G. Buse (1995) Diabetes 44 1438-1446).

WO 93/21330 (Zymogenetics) discloses DNA molecules encoding human glutamine:fructose-6-phosphate amidotransferase. The DNA molecules are used in methods to screen for glutamine:fructose-6-phosphate amidotransferase antagonists. The DNA molecules encoding human glutamine:fructose-6-phosphate amidotransferase are expressed in suitable host cells and recombinant glutamine:fructose-6-phosphate amidotransferase is produced. A test substance is exposed to the recombinant human glutamine:fructose-6-phosphate amidotransferase in the presence of fructose-6-phosphate and glutamine. A reduction in activity of the glutamine:fructose-6-phosphate amidotransferase in comparison to the activity in the absence of the test substance indicates a compound that inhibits human glutamine:fructose-6-phosphate amidotransferase.

U.S. Pat. No. 5,876,713 (Nishi et al.) discloses glutamine:fructose-6-phosphate amidotransferase; a DNA coding for the protein; a recombinant vector; a transformant; a method for producing the protein; a pharmaceutical composition comprising the protein; and an antibody against the protein or its partial peptide. The protein and the DNA are used as a prophylactic or therapeutic agent for hypoglycemia. The antibody is used in the assay of the protein and the protein is used as a reagent for the screening for candidate medical compounds.

### BRIEF DESCRIPTION OF THE FIGURES

FIG. 1 shows that the production of glucosamine 6-phosphate is dependent upon the amount of GFAT enzyme added to the reaction mixture. FIG. 1A is a graph illustrating the dependence of glucosamine 6-phosphate production upon the amount of GFAT-alpha enzyme. FIG. 1B is a graph illustrating the dependence of glucosamine 6-phosphate production upon the amount of GFAT-beta enzyme.

FIG. 2 is a graph illustrating that the levels of glucosamine 6-phosphate produced are dependent on the time of incubation with the GFAT enzyme.

FIG. 3 illustrates the effects of varying substrate concentrations, glutamine and fructose 6-phosphate, on the levels of glucosamine 6-phosphate produced by GFAT-alpha and GFAT-beta. FIG. 3A is a graph illustrating the results of incubating GFAT with 10 μl glutamine 200 mM+10 μl fructose 6-phosphate 3.125, 6.25, 12.5, 25, 50, 100, and 200 mM under standard incubation conditions at 37° C. for 60 minutes. FIG. 3B is a graph illustrating the results of incubating GFAT with 10 μl fructose 6-phosphate 200 mM+10 μl glutamine 3.125, 6.25, 12.5, 25, 50, 100, and 200 mM under standard incubation conditions at 37° C. for 60 minutes.

FIG. 3C is a table that illustrates the Km values of the substrates for the GFAT-alpha and GFAT-beta enzymes calculated from the data generated in FIGS. 3A and 3B.

FIG. 4 illustrates the determination of the optical density at 585 nm for known quantities of D-glucosamine 6-phosphate made in 50% DMSO at a concentration of 40 mM when added to the standard incubation mixture in the absence of active GFAT enzymes.

FIG. 5 is a table that summarizes the results of testing known GFAT inhibitors for their effects on GFAT-alpha and GFAT-beta enzymes.

FIG. 6 is a graph that illustrates the effects of acetylating glucosamine 6-phosphate with different concentrations of acetic anhydride.

FIG. 7 is a bar graph that illustrates the effects of time and temperature on glucosamine 6-phosphate acetylation in a microtiter plate determined by the optical density (OD) at 585 nm in the reaction mixture.

FIG. 8 is a bar graph illustrating a comparison of N-acetylglucosamine produced by different methods.

FIG. 9 is a graph that illustrates the effect of time and temperature on the hydrolysis of UDP-N-acetylglucosamine to N-acetylglucosamine.

FIG. 10 is a bar graph that illustrates a comparison of the determination of UDP-N-acetylglucosamine levels obtained with the HPLC/MS method and the acid hydrolysis method in extracts of COS cells transfected with GFAT-beta.

### SUMMARY OF THE INVENTION

The present invention relates to a method for measuring the activity of glutamine:fructose 6-phosphate amidotransferase, which comprises the steps of (a) forming a mixture of glutamine:fructose 6-phosphate amidotransferase, fructose 6-phosphate, and glutamine; (b) incubating the mixture in (a) under physiological conditions for a time sufficient to allow the formation of glucosamine 6-phosphate; (c) acetylating the glucosamine 6-phosphate in (b) to form N-acetylglucosamine 6-phosphate; (d) reacting the N-acetylglucosamine 6-phosphate in (c) with Ehrlich's reagent; and (e) measuring the amount of N-acetylglucosamine 6-phosphate present in (d) by determining the optical density at 500-610 nm of the mixture in (d) and comparing the amount of N-acetylglucosamine 6-phosphate in (d) with a control sample of N-acetylglucosamine 6-phosphate and a non-incubated control sample of glutamine:fructose 6-phosphate amidotransferase, or an incubated control sample of glutamine:fructose 6-phosphate amidotransferase without glutamine or without fructose 6-phosphate.

Preferably, the glutamine:fructose 6-phosphate amidotransferase is glutamine:fructose 6-phosphate amidotransferase-human alpha form having a Km of 1.49 mM for glutamine and a Km of 1.85 mM for fructose 6-phosphate or glutamine:fructose 6-phosphate amidotransferase-human beta form having a Km of 1.99 mM for glutamine and a Km of 6.8 mM for fructose 6-phosphate. The glutamine:fructose 6-phosphate amidotransferase may or may not be transfected into cells. Preferably, the mixture in (a) comprises glutamine:fructose 6-phosphate amidotransferase (0.25-25 μl), glutamine (2-40 mM), fructose 6-phosphate (2-40 mM), phosphate buffered saline pH 7.4, ethylenediaminetetraacetic acid (5 mM), and dithiothreitol (1 mM), the incubation step in (b) is carried out at 37° C. for a time period of 5 minutes to 5 hours, and the acetylation step in (c) is carried out with acetic anhydride 0.09375% in acetone followed by addition of potassium tetraborate 62.5 mM at 80° C. for 25 minutes. The Ehrlich's reagent in (d) preferably comprises 2 g p-dimethylaminobenzaldehyde, 0.3 ml water, 2.2 ml concentrated hydrochloric acid, and 17.4 ml acetic acid, which is diluted 1:2 in acetic acid. In a preferred embodiment, the mixture in (a) is formed in a microtiter plate and further comprises the step of adding a control sample of N-acetylglucosamine 6-phosphate and a non-incubated control sample of glutamine:fructose 6-phosphate amidotransferase, or an incubated control sample of glutamine:fructose 6-phosphate amidotransferase without glutamine or without fructose 6-phosphate to the microtiter plate, after the incubation step in (b).

The present invention also relates to a method for measuring the inhibitory activity of a test compound on glutamine:fructose 6-phosphate amidotransferase, which comprises the steps of (a) forming a mixture of a test compound, glutamine:fructose 6-phosphate amidotransferase, fructose 6-phosphate, and glutamine; (b) incubating the mixture in (a) under physiological conditions for a time sufficient to allow the test compound to inhibit glutamine:fructose 6-phosphate amidotransferase from forming glucosamine 6-phosphate; (c) acetylating the glucosamine 6-phosphate in (b) to form N-acetylglucosamine 6-phosphate; (d) reacting the N-acetylglucosamine 6-phosphate in (c) with Ehrlich's reagent; and (e) measuring the amount of N-acetylglucosamine 6-phosphate in (d) by determining the optical density at 500-610 nm of the mixture in (d) and comparing the amount of N-acetylglucosamine 6-phosphate in (d) with a control sample of glutamine:fructose 6-phosphate amidotransferase not containing the test compound and a control sample of N-acetylglucosamine 6-phosphate, thereby determining the inhibitory activity of the test compound.

Preferably, the mixture in (a) comprises test compound, glutamine:fructose 6-phosphate amidotransferase (0.25-25 μl), glutamine (10 mM), fructose 6-phosphate (10 mM), phosphate buffered saline pH 7.4, ethylenediaminetetraacetic acid (5 mM), and dithiothreitol (1 mM), the incubation step in (b) is carried out at 37° C. for 1 hour, and the acetylation step in (c) is carried out with acetic anhydride 0.09375% in acetone followed by addition of potassium tetraborate 62.5 mM at 80° C. for 25 minutes.

The present invention further relates to a method for measuring the formation of uridine-diphosphate-N-acetylglucosamine in extracts from human and animal tissue or cells in tissue cultures, which comprises the steps of (a) hydrolyzing an extract from human or animal tissue or cells in tissue culture to convert uridine-diphosphate-N-acetylglucosamine to N-acetylglucosamine; (b) reacting the N-acetylglucosamine in (a) with Ehrlich's reagent; and (c) measuring the amount of N-acetylglucosamine present in (b) by determining the optical density at 500-610 nm of the mixture in (b) and comparing the amount of N-acetylglucosamine in (b) with a control sample of N-acetylglucosamine, thereby determining the formation of uridine-diphosphate-N-acetylglucosamine.

Preferably, the extracts are from human tissue or cells. Preferably, the hydrolyzing step in (a) is carried out with hydrochloric acid 0.1N, followed by addition of potassium tetraborate 62.5 mM at 80° C. for 10 minutes and the Ehrlich's reagent in (b) comprises 2 g p-dimethylaminobenzaldehyde, 0.3 ml water, 2.2 ml concentrated hydrochloric acid, and 17.4 ml acetic acid, which is diluted 1:2 in acetic acid. In a preferred embodiment, the hydrolyzing step in (a) is carried out in a microfuge tube and the measuring step in (c) is carried out in a microtiter plate.

The present invention still further relates to a method for measuring the inhibitory activity of a test compound on uridine-diphosphate-N-acetylglucosamine formation in cells, which comprises the steps of (a) incubating a test compound with cells in tissue culture under physiological conditions for a time sufficient to allow the test compound to inhibit glutamine:fructose 6-phosphate amidotransferase and reduce cellular levels of uridine-diphosphate-N-acetylglucosamine; (b) extracting the uridine-diphosphate-N-acetylglucosamine in (a); (c) hydrolyzing the uridine-diphosphate-N-acetylglucosamine in (b) to form N-acetylglucosamine; (d) reacting the N-acetylglucosamine in (c) with Ehrlich's reagent; and (e) measuring the amount of N-acetylglucosamine in (d) by determining the optical density at 500-610 nm of the mixture in (d) and comparing the amount of N-acetylglucosamine in (d) with the amount in cells in tissue culture not incubated with the test compound and a control sample of N-acetylglucosamine, thereby determining the inhibitory activity of the test compound.

Preferably, the cells in (a) are selected from the group consisting of type 293 human kidney cells, type 293 human kidney cells transfected with GFAT-beta, or COS monkey kidney cells, COS monkey kidney cells transfected with GFAT-beta, mammalian cells, yeast cells, and bacterial cells. Preferably, the hydrolyzing step in (c) is carried out with hydrochloric acid 0.1N, followed by addition of potassium tetraborate 62.5 mM at 80° C. for 10 minutes and the Ehrlich's reagent in (d) comprises 2 g p-dimethylaminobenzaldehyde, 0.3 ml water, 2.2 ml concentrated hydrochloric acid, and 17.4 ml acetic acid, which is diluted 1:2 in acetic acid.

The present invention still further relates to a method for measuring the inhibitory activity of a test compound on uridine-diphosphate-N-acetylglucosamine formation in a human or an animal, which comprises the steps of (a) dosing a human or an animal with a test compound under physiological conditions for a time sufficient to allow the test compound to inhibit glutamine:fructose 6-phosphate amidotransferase from forming uridine-diphosphate-N-acetylglucosamine; (b) removing tissue from the human or animal in (a) and extracting the uridine-diphosphate-N-acetylglucosamine from the tissue; (c) hydrolyzing the uridine-diphosphate-N-acetylglucosamine in (b) to form N-acetylglucosamine; (d) reacting the N-acetylglucosamine in (c) with Ehrlich's reagent; and (e) measuring the amount of N-acetylglucosamine in (d) by determining the optical density at 500-610 nm of the mixture in (d) and comparing the amount of N-acetylglucosamine in (d) with the amount in a human or an animal not dosed with the test compound and a control sample of N-acetylglucosamine, thereby determining the inhibitory activity of the test compound.

Preferably, the dosing in (a) is carried out orally or intravenously 1 to 2 times per day over a period of 1 day to 2 weeks and the animal in (a) is a human, mouse, or rat. Preferably, the hydrolyzing step in (c) is carried out with hydrochloric acid 0. IN, followed by addition of potassium tetraborate 62.5 mM at 80° C. for 10 minutes.

### DETAILED DESCRIPTION OF THE INVENTION

The present invention relates to rapid spectrophotometric methods for high throughput microtiter plate analysis of glucosamine 6-phosphate levels. The methods also enable the simple measurement and quantitation of UDP-GlcNAc from a variety of sources. The spectrophotometric methods described are preferably performed in a microtiter plate and are based on the color reaction of N-acetylglucosamine with p-dimethylaminobenzaldehyde (Ehrlich's reagent) in dilute alkali solution. The spectrophotometric methods do not require the many purification steps needed for HPLC/MS methods and are capable of assaying many samples at a time. The methods include the hydrolysis of UDP-N-acetylglucosamine to N-acetylglucosamine, which is then measured by the spectrophotometric method for N-acetylglucosamine.

This invention is more fully described in copending patent application entitled “Methods for Measuring Activity of Glutamine:Fructose 6-Phosphate Amidotransferase And Activity of Inhibitors Thereof” filed by applicant concurrently with the present patent application and assigned to the assignee of this application, which is hereby incorporated by reference.

As used herein, the term “physiological conditions” refers to concentration, temperature, pH, ionic strength, viscosity, time, and various biochemical parameters which are compatible with a viable organism, and/or which typically exist intracellularly in a viable cultured animal cell or mammalian cell. Suitable reaction conditions for in vitro enzyme reactions are generally physiological conditions. The incubation of the mixture of glutamine:fructose 6-phosphate amidotransferase, fructose 6-phosphate, and glutamine is generally carried out under physiological conditions for a time sufficient to allow the formation of glucosamine 6-phosphate. For the measurement of glucosamine 6-phosphate, the preferred physiological conditions comprise NaCl 150 mM at pH 7.4 and 37° C. For the measurement of uridine-diphosphate-N-acetylglucosamine, the cells are preferably grown in Delbecco's Modified Eagles Medium (DMEM) at pH 7.4 with 10% fetal bovine serum. Particular aqueous conditions may be selected by the practitioner according to conventional methods with the optional addition of divalent cation(s) and/or metal chelators and/or non-ionic detergents and/or membrane fractions and/or anti-foam agents.

GFAT inhibitors reduce or inhibit the production of glucosamine 6-phosphate and ultimately the production of UDP-N-acetylglucosamine. The in vitro method using glutamine:fructose 6-phosphate amidotransferase only permits the measurement of glucosamine 6-phosphate. Another consideration in identifying a GFAT inhibitor of potential use in treatment of patients is whether the test compound enters cells in vivo. Under physiological conditions in cells, biological agents, e.g., enzymes, are produced in connection with the hexosamine biosynthetic pathway, which are involved in the production of UDP-GlcNAc. Compounds that are found to inhibit GFAT enzyme activity in cells can be tested in animals to determine if they enter the tissues, inhibit GFAT activity, and inhibit production of UDP-GlcNAc.

For measurement of N-acetylglucosamine 6-phosphate, the GFAT enzyme reaction and analysis of the product are preferably carried out directly in a microtiter plate. The preferred reaction volume of 100 μl consists of the substrates glutamine (10 MM) and fructose 6-phosphate (10 mM), phosphate buffered saline (PBS) pH 7.4, ethylenediaminetetraacetic acid (EDTA) (5 mM), dithiothreitol (DTT) (1 mM), and the GFAT enzyme preparation or tissue extract (0.25-50 μl). The incubation mixture is added to the plate, the plate sealed, and placed floating on a 37° C. water bath for 1 hour. Additional incubation mixture is kept on ice to add to standard and control samples. After incubation, non-incubated control and glucosamine 6-phosphate standards of 2.5 to 30 umoles along with the cold incubation mixture are added to the appropriate wells in the plate. The glucosamine 6-phosphate produced in the incubation mixtures is acetylated by first adding acetic anhydride 1.5% in acetone (10 μl), followed by potassium tetraborate 200 mM (50 μl). The plate is sealed, shaken for 2 minutes on a microshaker, and placed on an 80° C. water bath for 25 minutes. The plate is cooled by placing it on ice for 5 minutes and then Ehrlich's reagent is added (130 μl) (2 g Ehrlich's reagent +0.3 ml water+2.2 ml concentrated hydrochloric acid +17.4 ml acetic acid which is diluted 1:2 in acetic acid before use). The plate is then placed on a 37° C. water bath for 20 minutes and the OD determined at 500-610 nm, preferably 585 nm.

For measurement of UDP-N-acetylglucosamine in extracts from human and animal tissue or cells in tissue cultures, the extract sample is first hydrolyzed in 0.1N HCl at 80° C. for 10 minutes to produce N-acetylglucosamine. The sample is then cooled on ice for 5 minutes and neutralized with 0.1N KOH. Potassium tetraborate (62.5 mM[final]) is then added and the sample incubated at 80° C. for 25 minutes and then cooled on ice for 5 minutes. Ehrlich's reagent is then added and the sample is maintained for 20 minutes at 37° C. and then the OD is determined at 500-610 nm, preferably 585 nm.

### Stock Reagents for the Microtiter Plate Assay for Glucosamine 6-Phosphate

| | |
|---|---|
| Glutamine | 100 mM |
| Fructose 6-phosphate | 100 mM |
| PBS | 10X |
| EDTA | 50 mM |
| DTT | 10 mM |
| Acetic Anhydride | 1.5%/Acetone |
| Potassium Tetraborate | 200 mM |
| p-Dimethylaminobenzaldehyde | 2 g p-Dimethylaminobenzaldehyde/ |
| | 0.3 ml water |
| (Ehrlich's Reagent) | 2.2 ml conc. HCl/17.4 ml Acetic Acid |
| | diluted 1:2 in Acetic Acid before use |
| N-Acetylglucosamine | |
| for standards | |
| Enzyme Preparation or | |
| Tissue Extract | |

### Incubation Amounts for the Microtiter Plate Assay for Glucosamine 6-Phosphate

| | | |
|---|---|---|
| Glutamine 100 mM | 10 | ul |
| Fructose 6-phosphate 100 mM | 10 | ul |
| PBS 10X | 10 | μl |
| EDTA 50 mM | 10 | μl |
| DTT | 10 | μl |
| +/− Inhibitor | 10 | μl |
| Potassium Tetraborate 200 mM | 50 | μl |
| Ehrlich's reagent | 130 | μl |
| Enzyme Preparation or Tissue Extract | 0.25-50 | μl |
| Acetic Anhydride 1.5%/acetone | 10 | μl |
| Water | to 100 | μl |

### Procedure For Microtiter Plate Assay for Glucosamine 6-Phosphate

The reaction mixture is made including enough for standard curve samples and kept on ice. The reaction is started by adding the incubation reagents to the microtiter plate, sealing the plate, and placing the plate in a water bath at 37° C. for 1 hr. Standard curve samples are added to the plate, 2.5 to 30 nmoles/well/10 μl, and the cold mix added to control and standard curve samples. Glucosamine 6-phosphate is acetylated with acetic anhydride 1.5%/acetone 10 μl+potassium tetraborate 200 mM 50 μl, by sealing the plate, shaking for 2 minutes and placing on an 80° C. water bath for 25 minutes, then cooling on ice for 5 minutes. Diluted Ehrlich's reagent 130 μl is added and the plate placed on a 37° C. water bath for 20 minutes. The OD is determined at 585 nm.

Stock Reagents for the Spectrophotometric Assay for Measuring UDP-N-Acetylglucosamine

| | |
|---|---|
| HCl | 1N |
| KOH | 1N |
| Potassium Tetraborate | 200 mM |
| p-Dimethylaminobenzaldehyde | 2 g p-Dimethylaminobenzaldehyde/ |
| | 0.3 ml water |
| (Ehrlich's Reagent) | 2.2 ml conc. HCl/17.4 ml Acetic Acid |
| | diluted 1:2 in Acetic Acid before use |
| Acetonitrile | |
| N-Acetylglucosamine | |
| for standards | |
| Enzyme Preparation | |
| or Tissue Extract | |

### Incubation Mixture

| | | |
|---|---|---|
| +/− Inhibitor | 10 | μl |
| Acetonitrile (if needed) | 50 | μl |
| HCl 1 N | 10 | μl |
| KOH 1 N | 10 | μl |
| Potassium Tetraborate 200 mM | 50 | μl |
| Ehrlich's reagent | 150 | μl |
| Cells or Tissue Extract | 50-100 | μl |
| Water | to 100 | μl |

### Procedure For Spectrophotometric Assay for Measuring UDP-N-Acetylglucosamine

The reaction mixture is made as described above for the glucosamine 6-phosphate assay except the reaction is preferably started in a microfuge tube. If the sample contains too much protein, acetonitrile is added to first precipitate the protein to clarify the solution. The sample is then hydrolyzed in HCl 0.1N at 80° C. for 10 minutes, cooled on ice, and centrifuged 5 minutes to remove debris if there is high protein content. The sample is neutralized with KOH 0.1N. K₂B₄O₇ (59 mM [final]) is added and the sample incubated at 80° C. for 25 minutes an then cooled on ice for 5 minutes. Ehrlich's reagent is added for 20 minutes at 37° C., the samples centrifuged for 2 minutes, and 200 μl of the supernatant is added to a microtiter plate. The OD is determined at 585 nm.

Throughout this disclosure, applicant will suggest various theories or mechanisms by which applicant believes the present methods function. While applicant may offer various mechanisms to explain the present invention, applicant does not wish to be bound by theory. These theories are suggested to better understand the present invention but are not intended to limit the effective scope of the claims.

Throughout this application, various publications have been referenced. The disclosures in these publications are incorporated herein by reference in order to more fully describe the state of the art.

The present invention is further illustrated by the following examples that are presented for purposes of demonstrating, but not limiting, the preparation of the compounds and compositions of this invention.

### EXAMPLES

### High-Throughput Analysis of Glutamine:Fructose 6-Phosphate Amidotransferase Activity

### Example 1

### Measurement of Glucosamine 6-Phosphate

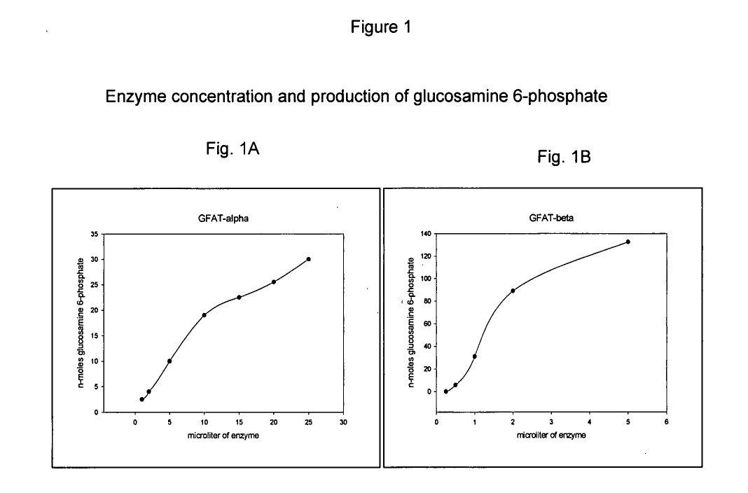

Enzyme preparation. GFAT-alpha (GenBank Accession No. M90516) and GFAT-beta (Accession No. AB016789) are cloned into pET12 and pEF-BOS C vectors (New England BioLabs) for transfection into SF9 and Hi5 insect cells, COS, type 293 cells, and E.coli. Endogenous activity for each cell line was also measured in non-transfected cells. The enzymes are expressed in each cell line by growing in large 24×24 cm plates and harvesting 20 hours after transfection. The media is removed and the plates are washed in 15 ml PBS. 1.5 ml of a lysis buffer (PBS 1×, KCl 50 mM, EDTA 10 mM, with protease inhibitors leupeptin 10 ug/ml, PMSF 20 ug/ml, A-protinin 30 ug/ml, and pepstatin 10 ug/ml) is added to each plate and the cells are removed using a large plastic cell scraper. This results in a final volume of about 3 ml. The cell #/lysis volume is about 4×10⁷cell/ml. The suspension is sonicated on ice (Branson sonicator setting 2.5 to 3) for 15 seconds using a microtip. The sonicated cell extracts are stored in microfuge tubes at −80° C. Before use, the extracts are centrifuged at 15000×g for 5 minutes at 4° C. Each enzyme preparation is titrated to determine the volume that gives between 20 to 30 umoles of glucosamine 6-phosphate in a reaction mixture in a 60-minute incubation at 37° C. (FIGS. 1A and 1B).

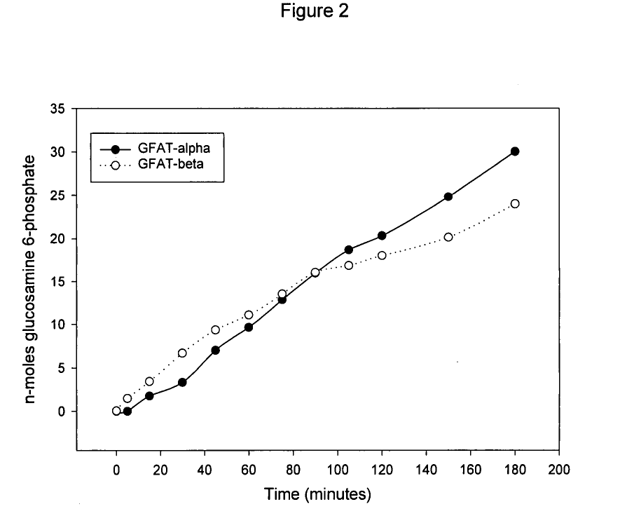

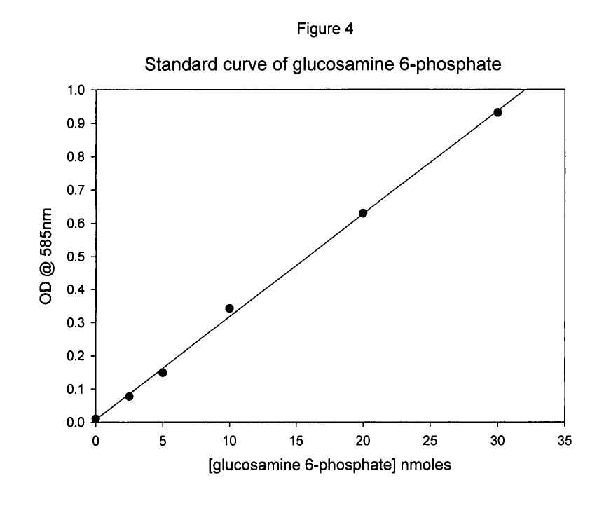

Stimulation of GFAT activity. The enzyme reaction was found to be linear over time (FIG. 2) in the presence of saturating substrate levels. For most testing, a 60-minute incubation at 37° C. is carried out in 96 well microtiter plates. To give the best heat transfer the plates are sealed with a Costar plate sealer and floated on a 37° C. water bath. Air bubbles are removed from the bottom of the plate to ensure proper heat transfer to all wells. The 100 μl reaction volume consists of glutamine 10 mM, fructose 6-phosphate 10 mM, PBS 1×, EDTA 5 mM, DTT 1 mM and the enzyme preparation. A mixture of the incubation reagents is kept on ice and the enzyme is added to the mixture just before addition to the plate. Additional incubation mixture is kept on ice to add to standard and control samples. The reaction did not take place at 4° C. If test agents are to be used, 10 μl is added to the appropriate wells and the vehicle is added to the control wells before addition of the incubation mixture. 15 wells are left blank for addition of control and standard glucosamine 6-phosphate samples. After the incubation, 10 μl of the standards made up in the appropriate vehicle (DMSO) are added to the appropriate wells. A concentration range of 2.5 to 30 umoles is in the linear portion of the curve and covers the quantity of glucosamine 6-phosphate produced under the incubation conditions (FIG. 4). The cold mixture is added to the standard sample wells and the glucosamine 6-phosphate is then measured.

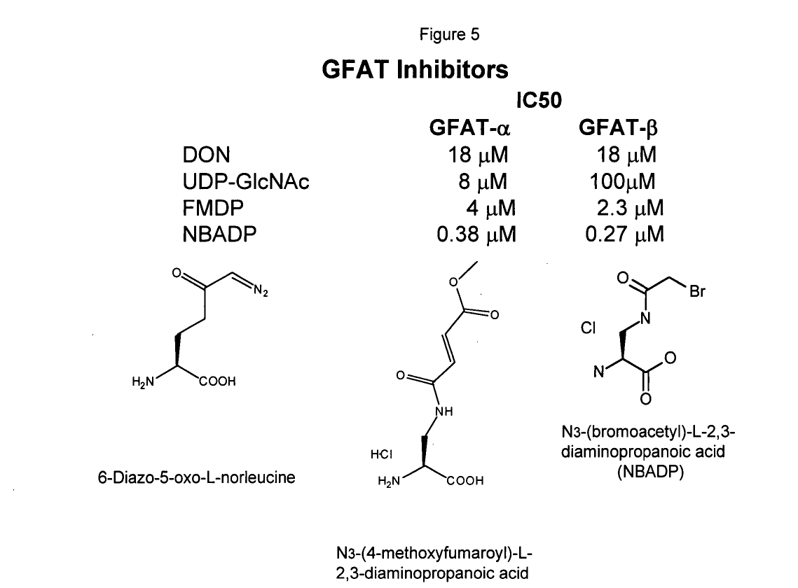

The activity of the GFAT-alpha and GFAT-beta enzymes expressed in SF9 insect cells were characterized with known inhibitors of GFAT (FIG. 5). DON (6-diazo-5-oxo-L-norleucine) inhibited both enzymes with an IC50 of 18 μM. UDP-N-acetylglucosamine inhibited GFAT-alpha with an IC50 of 8 μM, but more than 12 times as much UDP-N-acetylglucosamine was required to inhibit GFAT-beta having an IC50 of 100 μM. The glucosamine analog FMDP (N3-(-4-methoxyflumaroyl)-L-2,3-diaminopropanoic acid) inhibited GFAT-alpha with an IC50 of 4 μM and GFAT-beta with an IC50 of 2.3 uM. N3-(bromoacetyl)-L-2,3-diaminopropanoic acid inhibited GFAT-alpha, IC50 0.38 μM, and GFAT-beta, IC50 0.27 μM. The inhibition with UDP-N-acetylglucosamine is thus a discriminator between GFAT-alpha and GFAT-beta, and provides information about the functional roles of the two enzymes.

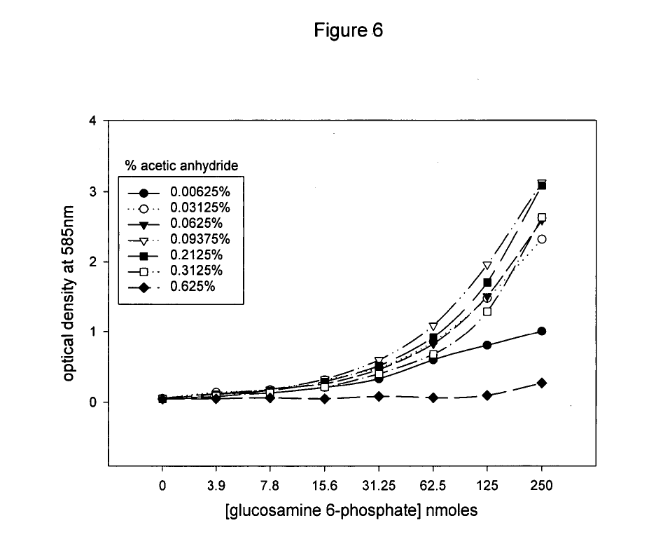

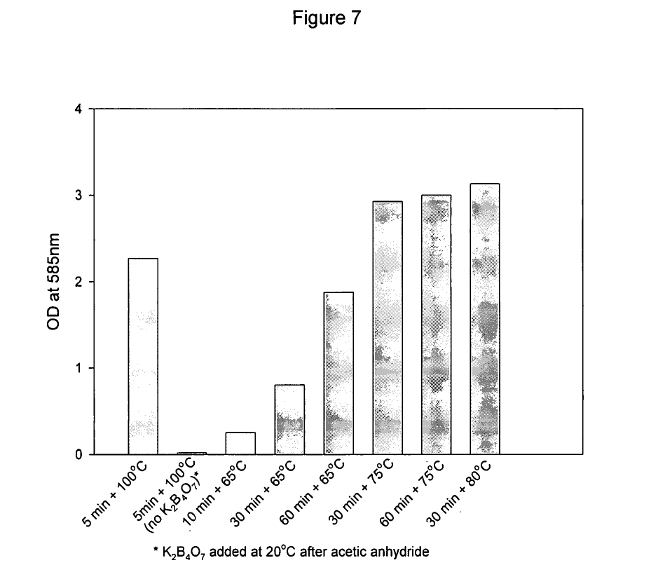

Measurement of glucosamine 6-phosphate The reaction is started by adding the incubation mixture to the appropriate wells in a microtiter plate and placing the plate on a 37° C. water bath. After the appropriate incubation time, the reaction is stopped by adding 10 μl of 1.5% acetic anhydride (final 0.09375%) followed by 50 μl of potassium tetraborate 200 mM (final 62.5 mM). The plate is sealed and placed on a micro-shaker for 2 minutes. The plate is then floated on an 80° C. water bath for 30 minutes taking care to remove air bubbles under the plate. 0.09375% acetic anhydride (out of a range of 0.00625% to 0.625%) at 80° C. (out of a range of 20° to 80° C.) was found to be the best concentration for acetylation of glucosamnine 6-phosphate (FIG. 6), and 30 minutes (out of a range of 10 to 30 minutes) at 80° C. (out of a range of 65° C. to 80° C.) to be a good incubation condition (FIG. 7). After the acetylation at 80° C., the plate is cooled on ice for 5 minutes. The acetylated glucosamine is measured by the Morgan and Elson color reaction (Morgan. W. T. J. & Elson, L. A. (1934).Biochem. J. 28, 988). 130 μl of Ehrlich's reagent (2 g p-dimethylaminobenzaldehyde+0.3 ml water+2.2 ml conc. HCl +17.4 ml acetic acid which is diluted 1:2 in acetic acid before use) is added to each well and incubated at 37° C. by floating on a water bath for 20 minutes. The OD is determined at 585 nm and the quantity of glucosamine 6-phosphate produced is calculated from the standard curve using a Softmax-Pro program to interpolate the ODs from the standard curve to give the moles produced.

### Example 2

### Measurement of UDP-N-Acetylglucosamine in Cells and Tissue

Tissue extract. Tissue to be assayed is kept frozen at −80° C. and kept on dry ice during the preparation and weighing process. The tissue is pulverized to a fine powder by rapping the tissue in heavy foil, placing it on top of a metal block in a bed of dry ice, and rapidly hammering it until it is a fine powder. It is then placed in a tarred microfuge tube and kept on dry ice until weighed. The tissue is extracted in chloroform:water (1 mg+2 μl chloroform+2 μl water). The suspension is sonicated for about 5 seconds on ice (Branson sonicator setting 3 to 5 with micro-tip) to breakup any clumps. The extract can then be stored at −80° C. Before analysis, the tissue extract is then thawed and centrifuged at 15000×g for 20 minutes at 4° C.

Cell extract: Cells in culture are incubated in 35 mm culture dishes with and without a GFAT inhibitor and treated with glucose to produce UDP-N-acetylglucosamine. Cells are normally grown to about 80% confluency in DMEM-glucose 4.5 mM +10% fetal bovine serum. The media is then changed to DMEM (no glucose) +10% fetal bovine serum for 12 to 16 hours. If a GFAT inhibitor is present, it is added for 30 minutes, and followed by addition of glucose 25 mM for time periods of 1 hour to 24 hours. The media is removed, cells washed 1× in PBS, and extracted in 200 μl of chloroform:water (1:1). The suspension is sonicated for about 5 seconds on ice to breakup any clumps. The extract can be stored at −80° C. Before analysis, the tissue extract is then thawed and centrifuged at 15000×g for 20 minutes at 4° C.

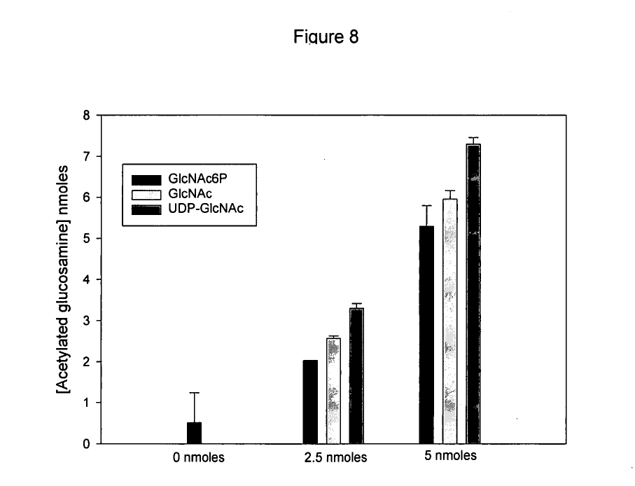

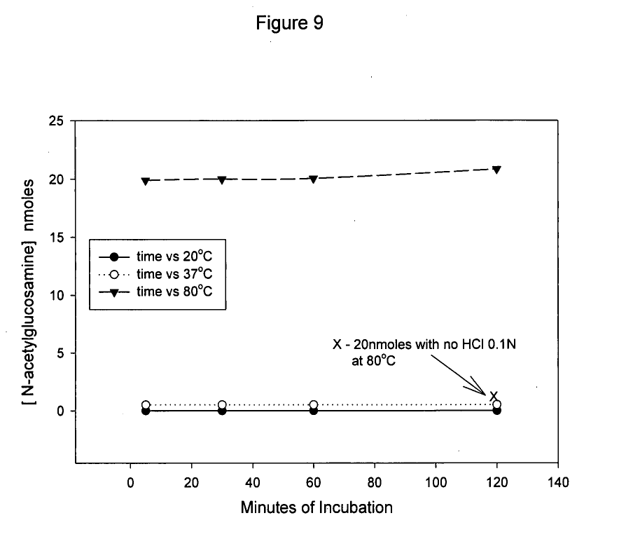

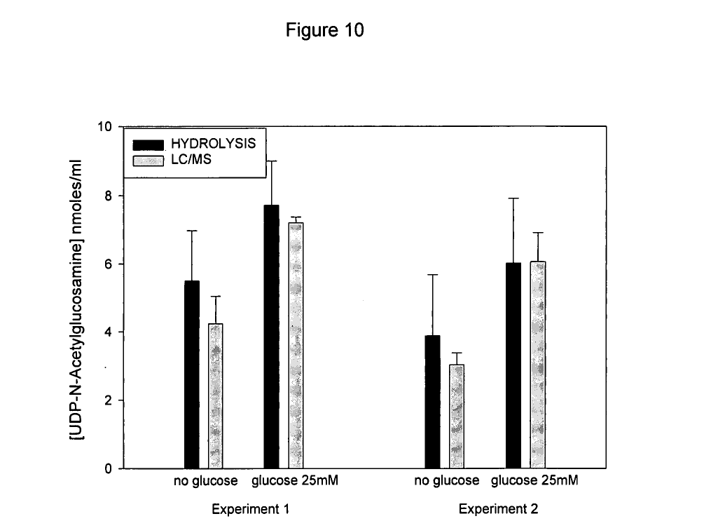

Analysis of UDP-N-Acetylglucosamine in tissue extracts. 50 μl of tissue extract (25 mg starting tissue) is placed in a microfuge tube and 50 μl of acetonitrile is added. This is needed to precipitate excess protein in the sample and help clarify the sample. To hydrolyze the UDP-N-acetylglucosamine in the sample, 10 μl of HCl IN is added and the sample vortexed. This mixture is heated at 80° C. for 20 minutes. The sample is then centrifuged 15000×g at room temperature for 15 seconds to precipitate any condensation formed. The sample is neutralized with the addition of 10 μl KOH 1N. The resulting product, N-acetylglucosamine, is measured by the Morgan and Elson color reaction. 50 μl of potassium tetraborate 200 mM is added and the sample heated at 80° C. for 25 minutes followed by cooling on ice for 5 minutes. 150 μl of Ehrlich's reagent is added and mixed. After 20 minutes at 37° C., the sample is centrifuged 15000×g at room temperature for 2 minutes. 200 μl of the supernatant is added to a 96 well plate and the OD determined at 585 nm. The quantity of N-acetylglucosamine is calculated from standard samples of N-acetylglucosamine using a Softmax-Pro program. The standard curve samples are prepared from N-acetylglucosamine, 2.5 to 30 nmoles/well, in a similar manner to the tissue samples. Comparison experiments were performed to confirm the analysis of N-acetylglucosamine produced by three different methods. The experiments demonstrated that: 1) glucosamine 6-phosphate acetylated with acetic anhydride+potassium tetraborate, 2) N-acetylglucosamine treated with potassium tetraborate, and 3) UDP-N-acetylglucosamine hydrolyzed with HCl 0.1N followed by treatment with potassium tetraborate all gave similar results (FIG. 8). The data also demonstrates that the UDP-N-acetylglucosamine is completely hydrolyzed to N-acetylglucosamine with HCl 0.1N after 20 minutes at 80° C. Studies on time (5 to 120 minutes) and temperature (20, 37, and 80° C.) indicate that 5 minutes at 80° C. is sufficient for complete hydrolysis of UDP-N-acetylglucosamine to N-acetylglucosamine (FIG. 9). These studies also indicate that UDP-N-acetylglucosamine treated with potassium tetraborate does not react with Ehrlich's reagent to produce a color. To verify that the hydrolysis method and the HPLC/MS method give comparable results extracts of COS cells were analyzed by the two methods. COS cells transfected with GFAT-beta grown in glucose free media for 16 hours were exposed to glucose 25 mM for 5 hours. Glucose treatment results in an increase in cellular UDP-N-acetylglucosamine levels. The measured levels of no glucose and glucose 25 mM stimulated cells were comparable between the two methods (FIG. 10).

FIG. 1 shows that the production of glucosamine 6-phosphate is dependent upon the amount of GFAT enzyme. FIG. 1A is a graph illustrating the dependence of glucosamine 6-phosphate production upon the GFAT-alpha enzyme concentration. FIG. 1B is a graph illustrating the dependence of glucosamine 6-phosphate production upon the GFAT-beta enzyme concentration. To an incubation mixture of 10 μl glutamine 100 mM, 10 μl fructose 6-phosphate 100 mM, 10 μl PBS 10×, 10 μl EDTA 50 mM, 10 μl DTT 10 mM, and water to 100 μl is added 1, 2, 5,10, 15, 20, and 25 μl of GFAT-alpha cell extracts, or 0.25, 0.5, 1, 2, and 5 μl of GFAT-beta cell extracts. The incubation is started by adding the incubation mixture to the appropriate wells of a 96 well microtiter plate, separate plates are used for each enzyme, and placing (floating) the plates on a 37° C. water bath. After a 60-minute incubation, the plates are removed from the water bath and the standard curve samples are added to the appropriate wells of the plate. This is followed by the addition of 10 μl of acetic anhydride 1.5% and 50 μl potassium tetraborate 200 mM to all the wells and shaking the plate for 2 minutes at room temperature on a microshaker. The plates are incubated at 80° C. by placing the plates (floating) on an 80° C. water bath for 30 minutes. The plates are cooled on ice for 5 minutes. 130 μl of Ehrlich's reagent is added to all the wells for 20 minutes at 37° C. and the OD determined for all of the wells at 585 nm. The n-moles of glucosamine 6-phosphate in each well is determined for each concentration of enzyme.

The differentiation of the GFAT-alpha from the GFAT-beta can be determined by their sensitivity to UDP-N-acetylglucosamine. GFAT-alpha is inhibited by UDP-N-acetylglucosamine with an IC50 of 8 μM. GFAT-beta is inhibited by UDP-N-acetylglucosamine with an IC50 of 100 μM.

FIG. 2 is a graph illustrating that GFAT activity is dependent on the time of incubation as expressed in Hi5 insect cells. To determine the GFAT activity over time extracts of GFAT-alpha and GFAT-beta expressed in Hi5 insect cells are incubated over a 180-minute period. The reaction is done in 96 well microtiter plates. The incubation mixtures are 1) 10 μl glutamine 100 mM, 10 μl fructose 6-phosphate 100 mM, 10 μl PBS 10×, 10 μl EDTA 50 mM, 10 μl DTT 10 mM, 5 μl GFAT-alpha Hi5 cell extract, and 35 μl water, and 2) 10 μl glutamine 100 mM, 10 μl fructose 6-phosphate 100 mM, 10 μl PBS 10×, 10 μl EDTA 50 mM, 10 μl DTT 10 mM, 0.25 μl GFAT-beta Hi5 cell extract, and 39.75 μl water. The reaction mixtures are kept on ice and the GFAT-alpha and GFAT-beta enzymes incubated in different plates. The reaction is started by adding 100 μl of the incubation mixture to the plate starting with the 180 minute time with the 0 time last. The reaction is stopped by placing the plate on ice. The standard samples are added to the appropriate wells of the plate followed by addition of 10 μl of acetic anhydride 1.5% in acetone and 50 μl of potassium tetraborate 200 mM. The plates are shaken for 2 minutes at room temperature and are incubated at 80° C. for 30 minutes by floating on a water bath. The plates are then placed on ice for 5 minutes followed by addition of Ehrlich's reagent 130 μ. After a 20-minute incubation at 37° C., the OD at 585 nm is determined for all the wells on the plate and the quantity of N-acetyl glucosamine 6-phosphate determined. Similar results are obtained with GFAT enzymes prepared from different sources.

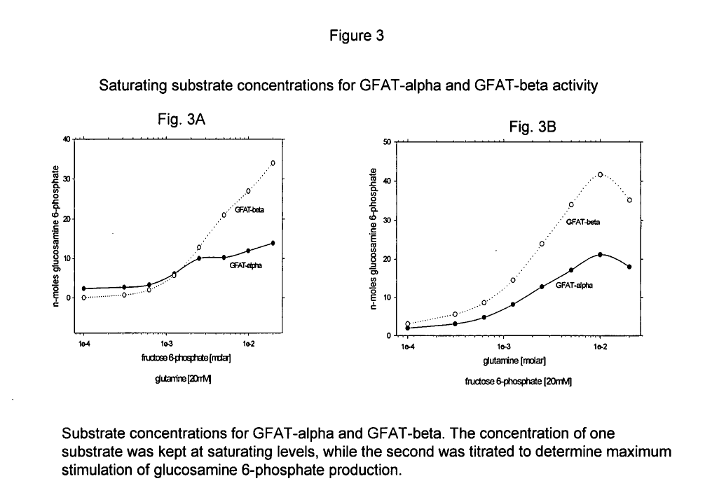

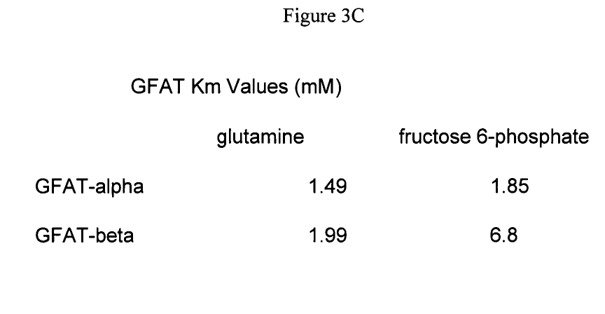

FIG. 3 illustrates the effect of varying concentrations of the substrates glutamine and fructose 6-phosphate on the enzyme activity of GFAT-alpha and GFAT-beta. FIG. 3A is a graph illustrating incubation with 10 μl glutamine 200 mM+10 μl fructose 6-phosphate 3.125, 6.25, 12.5, 25, 50, 100, and 200 mM. FIG. 3B is a graph illustrating incubation with 10 μl fructose 6-phosphate 200 mM+10 μl glutamine 3.125, 6.25, 12.5, 25, 50, 100, and 200 mM. 10 μl of GFAT-alpha and 5 μl of GFAT-beta extracts from transfected SF9 insect cells are incubated with 1) 10 μl glutamine 200 mM+10 μl fructose 6-phosphate 3.125, 6.25, 12.5, 25, 50, 100, and 200 mM, and 2) 10 μl fructose 6-phosphate 200 mM+10 μl glutamine 3.125, 6.25, 12.5, 25, 50, 100, and 200 mM in an incubation media containing 10 μl PBS 10×, 10 μl EDTA 50 mM, 10 μl DTT 10 mM, and water to 100 μl. The plate is sealed with a Costar plate sealer and incubated for 60 minutes at 37° C. by floating on a water bath. Additional incubation mixture is kept on ice to add to standard and control samples. After the incubation, standards along with the cold incubation mixture are added to the appropriate wells on the plate, followed by 10 μl acetic anhydride and 50 μl potassium tetraborate 200 mM to all the wells. The plate is shaken for 2 minutes and incubated at 80° C. by floating on a water bath. The plate is cooled on ice for 5 minutes followed by addition of Ehrlich's reagent to each well. After a 20-minute incubation at 37° C., the OD is determined for each well at 585 nm.

FIG. 3C is a table illustrating the Km values of the substrates for the GFAT-alpha and GFAT-beta enzymes calculated from the data generated in FIGS. 3A and 3B. A plot of the data using a Lineweaver-Burk plot (Lineweaver, H. and Burk, D (1934) J.Amer.Chem.Soc. 56, 658-666) gave the Km values reported. In the table, the Km of glutamine for GFAT-alpha and GFAT-beta are 1.49 mM and 1.99 mM, respectively. The Km of fructose 6-phosphatefor for GFAT-beta is 6.8 mM and for GFAT-alpha is 1.85 mM.

FIG. 4 is a graph illustrating the standards of D-glucosamine 6-phosphate (Sigma-Aldrich Cat# G5509) made in 50% DMSO at a concentration of 40 mM. Dilutions are made from this stock solution to give 0, 2.5, 5, 10, 20, and 30 nmoles in a 10 μl sample. The OD of the 0 mM, DMSO vehicle alone, is subtracted from all of the determined values on the plate. 10 μl of the standard samples are added to the appropriate wells on the plate, in duplicate wells. The plate is sealed with a Costar plate sealer and incubated for 60 minutes at 37° C. by floating on a water bath. During the incubation the incubation mixture is kept on ice. The incubation mixture consists of 10 μl glutamine 100 mM, 10 μl fructose 6-phosphate 100 mM, 10 μl PBS 10×, 10μl EDTA 50 mM, 10μl DTT 10 mM, 2 μl of an enzyme extract of COS cells transfected with GFAT-alpha, and 48 μl water. After the incubation 90 μl of the cold incubation mixture is added. The reaction is stopped by the addition of 10 μl acetic anhydride 1.5% followed by 50 μl of potassium tetraborate 200 mM to all the wells. The plate is shaken for 2 minutes and incubated for 30 minutes at 80° C. by floating on a water bath. The plate is cooled on ice for 5 minutes followed by the addition of Ehrlich's reagent to all the wells. After a 20-minute incubation at 37° C., the OD is determined for all the wells at 585 nm.

FIG. 5 illustrates the testing of known inhibitors of GFAT for sensitivity against the GFAT-alpha and GFAT-beta enzymes. The incubation mixture consists of 10 μl glutamine 100 mM, 10 μl fructose 6-phosphate 100 mM, 10 μl PBS 10×, 10 μl EDTA 50 mM, 10 μl DTT 10 mM, 51 μl GFAT-alpha or 0.25 μl GFAT-beta, 10 μl of the test compound in DMSO 50% or DMSO 50% alone, and water to bring the final volume to 10 μl. The compounds are added to the appropriate wells of a 96 well microtiter plate followed by 90 μl of the incubation media for GFAT-alpha and GFAT-beta to the appropriate wells. The plate is incubated at 37° C. by placing the plate on a water bath for 60 minutes. The standard samples are then added to the plate followed by 10 μl of acetic anhydride 1.5% and 50 μl potassium tetraborate 200 mM to all the wells. After shaking the plate for 2 minutes, the plate is incubated at 80° C. for 30 minutes. The plate is cooled on ice for 5 minutes and 130 μl Ehrlich's reagent is added for 20 minutes at 37° C. to all the wells. The OD for each well is determined at 585 nm and the amount of glucosamine 6-phosphate produced is calculated. The amount of inhibition with the compound is determined from the enzyme activity in the presence of DMSO alone. The inhibition is expressed as the IC50 or the concentration inhibiting 50% of maximum stimulation.

FIG. 6 is a graph illustrating the acetylation of glucosamine 6-phosphate with different concentrations of acetic anhydride. The reaction is carried out in a 96 well microtiter plate. The incubation mixture consists of glucosamine 6-phosphate 1 μmole in 100 μl+10 μl of acetic anhydride made up in acetone at initial concentrations of 0.1% to 10% which gives final concentrations of 0.00625% to 0.625%+50 μl potassium tetraborate 200 mM. The mixture is incubated at 80° C. for 30 minutes by sealing the plate with a Costar plate sealer and floating the plate on an 80° C. water bath. After the incubation, the plate is cooled on ice for 5 minutes. 130 μl of Ehrlich's reagent is added to all the wells, the plate sealed, and the plate is incubated at 37° C. for 20 minutes by floating on a 37° C. water bath. The OD is determined for all the wells at 585 nm and the data is expressed as the optical density produced at the different concentrations of acetic anhydride.

FIG. 7 is a bar graph illustrating the optimum conditions for acetylation of glucosamine 6-phosphate in wells of a microtiter plate by varying times and temperatures. To permit the assay to be performed in a microtiter plate temperatures less than 100° C. are necessary. The incubations are performed in microfuge tubes in this assay for ease of handling the different conditions. 1 mole of glucosamine 6-phosphate in 100 μl water+22.5 μl acetic anhydride 1.5% in acetone +50 μl potassium tetraborate 200 mM+80 μl water are incubated under the different conditions. After each incubation, the tube is cooled on ice for 5 minutes and 1400 μl of Ehrlich's reagent is added to each tube for 20 minutes at 37° C. The OD is determined at 585 nm. The following incubation conditions are performed: 1) As a standard, a 5 minute incubation of the acetylation mixture is placed in a boiling water bath for 5 minutes. 2) A 5-minute incubation with acetic anhydride alone at 100° C. followed by potassium tetraborate for 5 minutes at room temperature is done to demonstrate the necessity of the heating step with potassium tetraborate. 3) A 10-minute incubation at 65° C. 4) A 30-minute incubation at 65° C. 5) A 60-minute incubation at 65°. 6) A 30-minute incubation at 75° C. 7) A 60-minute incubation at 75° C. 8) A 30-minute incubation at 80° C. As the temperature increases, the OD increases indicating better sensitivity. As the time is increased, the OD increases indicating better sensitivity.

FIG. 8 is a bar graph illustrating a comparison of N-acetylglucosamine produced by different methods. Standard solutions of glucosamine 6-phosphate (Sigma-Aldrich G5509), N-acetylglucosamine (Sigma-Aldrich A8625), and UDP-N-acetylglucosamine (Sigma-Aldrich U4375) are dissolved in water to a concentration of 4 mM and diluted to 2.5 and 5 nmoles/10 μl. The standard solutions are treated in the following manner: 1) Glucosamine 6-phosphate is acetylated by adding 10 μl of each solution to 90 μl water, 10 μl HCl IN, 10 μl KOH 1N, and 10 μl acetic anhydride 1.5% followed by 50 μl potassium tetraborate 200 mM. The mixture is heated to 80° C. in a water bath for 30 minutes and then cooled on ice for 5 minutes. 2) 10 μl of the N-acetylglucosamine solutions are added to 90 μl water, 10 μl HCl 1N, 10 μl KOH 1N, and 50 μl potassium tetraborate 200 mM. The mixture is heated at 80° C. in a water bath for 30 minutes and then cooled on ice for 5 minutes. 3) 10 μl of each standard solution of UDP-N-acetylglucosamine is added to 90 μl water and 10 μl HCl 1N. This mixture is hydrolyzed by heating in an 80° C. water bath for 10 minutes and then centrifuged 15000×g for 15 seconds. 10 μl KOH 1N and 50 μl potassium tetraborate 200 mM are added to each tube and heated at 80° C. for 30 minutes in a water bath. The reaction is cooled on ice for 5 minutes. 150 μl of Ehrlich's reagent is added to the acetylated products and analyzed as described in methods.

FIG. 9 is a graph illustrating the effect of time and temperature on the hydrolysis of UDP-N-acetylglucosamine to N-acetylglucosamine. To determine the temperature and time necessary for hydrolysis of UDP-N-acetylglucosamine to N-acetylglucosamine, 20 nmoles of UDP-N-acetylglucosamine are incubated with HCl 0.1N at 20, 37, and 80° C. for 5, 30, 60 and 120 minutes. 10 μl of UDP-N-acetylglucosamine +10 μl HCl 1N+80 μl water are incubated in a microfuge tube for the times and temperatures indicated. 50 μl potassium tetraborate 200 mM is added for 30 minutes at 80° C. followed by cooling on ice for 5 minutes. 130μl Ehrlich's reagent is added to each tube for 20 minutes at 37° C. and the samples transferred to a 96 well microtiter plate to determine the OD at 585 nm. A sample with 20 nmoles of UDP-N-acetylglucosamine and no HCl 0.1N did not produce any N-acetylglucosamine indicating that HCl, or another suitable acidic solution, is required for hydrolysis of UDP-N-acetylglucosamine. Since no product was formed at 20° C. or 37° C., these results indicate that a temperature greater than 37° C. is required.

FIG. 10 is a bar graph illustrating a comparison of the levels of UDP-N-acetylglucosamine obtained with the HPLC/MS method and the hydrolysis method described herein on extracts of COS cells transfected with GFAT-beta. The COS-GFAT-beta cells are plated on 35 mm plates and when they reach 80% confluency the DMEM media is changed to DMEM plus 10% fetal bovine serum with no glucose for 16 hours. Glucose to a final concentration of 25 mM is then added for 5 hours. The media is aspirated, the cells are washed in PBS 1×, scraped in 0.5 ml of PBS, and transferred to a microfuge tube. The cells are centrifuged 2 minutes at 1500×g at 4° C., and the pellet extracted in 200 μl water+200 μl chloroform. The extract is sonicated for 15 seconds on ice using a microtip, and then centrifuged at 1500×g for 5 minutes at 4° C. and the supernatant saved. 10 μl of the extract is taken for analysis by the HPLC/MS method. 150 μl of the extract is placed in a microfuge tube +50 μl acetonitrile +20μl HCl 1N. This mixture is heated to 80° C. for 10 minutes. 20 μl KOH 1N is added +50 μl potassium tetraborate 200 mM to each tube and heated at 80° C. for 30 minutes, followed by cooling on ice for 5 minutes. 150 μl of Ehrlich's reagent is added to each tube for 20 minutes at 37° C. and 300 μl is transferred to a 96 well microtiter plate for OD determination at 585 nm. The n-moles of N-acetylglucosamine are calculated from a standard curve of N-acetylglucosamine.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

What is claimed is:

1. A method for measuring the formation of uridine-diphosphate-N-acetylglucosamine in extracts from human and animal tissue or cells in tissue cultures, which comprises the steps of:
(a) hydrolyzing an extract from human or animal tissue or cells in tissue culture to convert uridine-diphosphate-N-acetylglucosamine to N-acetylglucosamine;
(b) reacting the N-acetylglucosamine in (a) with Ehrlich's reagent; and
(c) measuring the amount of N-acetylglucosamine present in (b) by determining the optical density at 500-610 nm of the mixture in (b) and comparing the amount of N-acetylglucosamine in (b) with a control sample of N-acetylglucosamine, thereby determining the formation of uridine-diphosphate-N-acetylglucosamine.

2. The method according to claim 1, wherein the extracts are from human tissue or cells.

3. The method according to claim 1, wherein the hydrolyzing step in (a) is carried out with hydrochloric acid, followed by addition of potassium tetraborate for 10 to 30 minutes.

4. The method according to claim 3, wherein the hydrolyzing step in (a) is carried out with hydrochloric acid, followed by addition of potassium tetraborate for 10 minutes.

5. The method according to claim 3, wherein the hydrolyzing step in (a) is carried out with hydrochloric acid, followed by addition of potassium tetraborate at greater than 37° C.

6. The method according to claim 1, wherein the Ehrlich's reagent in (b) comprises 2 g p-dimethylaminobenzaldehyde, 0.3 ml water, 2.2 ml concentrated hydrochloric acid, and 1 7.4 ml acetic acid, which is diluted 1:2 in acetic acid.

7. The method according to claim 1, wherein the hydrolyzing step in (a) is carried out in a microfuge tube.

8. The method according to claim 1, wherein the measuring step in (c) is carried out in a microtiter plate.

9. A method for measuring the inhibitory activity of a test compound on uridine-diphosphate-N-acetylglucosamine formation in cells, which comprises the steps of:
(a) incubating a test compound with cells in tissue culture under physiological conditions for a time sufficient to allow the test compound to inhibit glutamine:fructose 6-phosphate amidotransferase and reduce cellular levels of uridine-diphosphate-N-acetylglucosamine;
(b) extracting the uridine-diphosphate-N-acetylglucosamine in (a);
(c) hydrolyzing the uridine-diphosphate-N-acetylglucosamine in (b) to form N-acetylglucosamine;
(d) reacting the N-acetylglucosamine in (c) with Ehrlich's reagent; and
(e) measuring the amount of N-acetylglucosamine in (d) by determining the optical density at 500-610 nm of the mixture in (d) and comparing the amount of N-acetylglucosamine in (d) with the amount in cells in tissue culture not incubated with the test compound and a control sample of N-acetylglucosamine, thereby determining the inhibitory activity of the test compound.

10. The method according to claim 9, wherein the cells in (a) are selected from the group consisting of type 293 human kidney cells, type 293 human kidney cells transfected with GFAT-beta, or COS monkey kidney cells, COS monkey kidney cells transfected with GFAT-beta, mammalian cells, yeast cells, and bacterial cells.

11. The method according to claim 9, wherein the hydrolyzing step in (c) is carried out with hydrochloric acid, followed by addition of potassium tetraborate for 10 to 30 minutes.

12. The method according to claim 11, wherein the hydrolyzing step in (c) is carried out with hydrochloric acid, followed by addition of potassium tetraborate for 10 minutes.

13. The method according to claim 11, wherein the hydrolyzing step in (c) is carried out with hydrochloric acid, followed by addition of potassium tetraborate at greater than 37°.

14. The method according to claim 9, wherein the Ehrlich's reagent in (d) comprises 2 g p-dimethylaminobenzaldehyde, 0.3 ml water, 2.2 ml concentrated hydrochloric acid, and 17.4 ml acetic acid, which is diluted 1:2 in acetic acid.

15. The method according to claim 9, wherein the hydrolyzing step in (c) is carried out in a microfuge tube.

16. The method according to claim 9, wherein the measuring step in (e) is carried out in a microtiter plate.

17. A method for measuring the inhibitory activity of a test compound on uridine-diphosphate-N-acetylglucosamine formation in a human or an animal, which comprises the steps of:
(a) dosing a human or an animal with a test compound under physiological conditions for a time sufficient to allow the test compound to inhibit glutamine:fructose 6-phosphate amidotransferase from forming uridine-diphosphate-N-acetylglucosamine;
(b) removing tissue from the human or animal in (a) and extracting the uridine-diphosphate-N-acetylglucosamine from the tissue;
(c) hydrolyzing the uridine-diphosphate-N-acetylglucosamine in (b) to form N-acetylglucosamine;
(d) reacting the N-acetylglucosamine in (c) with Ehrlich's reagent; and
(e) measuring the amount of N-acetylglucosamine in (d) by determining the optical density at 500-610 nm of the mixture in (d) and comparing the amount of N-acetylglucosamine in (d) with the amount in a human or an animal not dosed with the test compound and a control sample of N-acetylglucosamine, thereby determining the inhibitory activity of the test compound.

18. The method according to claim 17, wherein the dosing in (a) is carried out orally or intravenously 1 to 2 times per day over a period of 1 day to 2 weeks.

19. The method according to claim 17, wherein the animal in (a) is a human, mouse, or rat.

20. The method according to claim 17, wherein the hydrolyzing step in (c) is carried out with hydrochloric acid 0.1N, followed by addition of potassium tetraborate for 10 to 30 minutes.

21. The method according to claim 17, wherein the hydrolyzing step in (c) is carried out with hydrochloric acid, followed by addition of potassium tetraborate for 10 minutes.

22. The method according to claim 17, wherein the hydrolyzing step in (c) is carried out with hydrochloric acid, followed by addition of potassium tetraborate at greater than 37° C.

23. The method according to claim 17, wherein the Ehrlich's reagent in (d) comprises 2 g p-dimethylaminobenzaldehyde, 0.3 ml water, 2.2 ml concentrated hydrochloric acid, and 17.4 ml acetic acid, which is diluted 1:2 in acetic acid.

24. The method according to claim 17, wherein the hydrolyzing step in (c) is carried out in a microfuge tube.

25. The method according to claim 17, wherein the measuring step in (e) is carried out in a microtiter plate.
